(19) United States
(12) Patent Application Publication
(10) Pub. No.: US 2025/0127510 A1
(43) Pub. Date: Apr. 24, 2025

(54) PREFERENTIALLY BENDING MICROSTRUCTURES FOR MECHANICAL ADHESION

(71) Applicant: The Charles Stark Draper Laboratory, Inc., Cambridge, MA (US)

(72) Inventors: Daniel Frederick KING, Watertown, MA (US); Corin WILLIAMS, Framingham, MA (US); David J. CARTER, Concord, MA (US); Joseph URBAN, Cambridge, MA (US); Beau LANDIS, Cambridge, MA (US)

(73) Assignee: The Charles Stark Draper Laboratory, Inc., Cambridge, MA (US)

(21) Appl. No.: 18/922,876

(22) Filed: Oct. 22, 2024

Related U.S. Application Data
(60) Provisional application No. 63/545,267, filed on Oct. 23, 2023.

(51) Int. Cl. A61B 17/08; A61F 2/24

(52) U.S. Cl. CPC A61B 17/08; A61F 2/2418; A61F 2210/0014; A61F 2220/0016; A61F 2250/0082

(57) ABSTRACT

A micro-structured mechanical adhesive that can be used as an alternative for suturing is provided. The micro-structured mechanical adhesive includes a base section having a first side and a second side and a plurality of microstructures extending from the first side of the base section. Characteristically, each microstructure includes a preferential failure notch that biases the microstructure to preferentially yield or buckle in a predetermined direction. Advantageously, the micro-structured mechanical adhesive is configured to provide attachment to biological tissue.

### CROSS-REFERENCE TO RELATED APPLICATIONS

This application claims the benefit of U.S. provisional application Ser. No. 63/545,267 filed Oct. 23, 2023, the disclosure of which is hereby incorporated in its entirety by reference herein.

### STATEMENT REGARDING FEDERALLY SPONSORED RESEARCH OR DEVELOPMENT

This invention was made with Government support under Contract No. W81-XWH-20-1-0295 awarded by the Department of Defense. The Government has certain rights in the invention.

### TECHNICAL FIELD

In at least one aspect, the present invention is related to microstructured adhesive surfaces.

### BACKGROUND

The state-of-the-art for wound closure and attachment of medical devices to tissue is suturing by hand. Suturing requires significant skill, leading to extended times in surgery as well as high manufacturing costs for devices that require the integration of two unlike materials (e.g., metal and soft tissue grafts, such as are currently used in bioprosthetic heart valves). As an example, the assembly of bioprosthetic heart valves currently requires suturing by hand, a labor-intensive process performed by highly trained technicians. In this process, the tissue cannot be fully punctured in order to avoid leakage and failure upon implantation.

Bio-adhesives can provide an alternative to conventional suturing. However, there are several hurdles to overcome, such as adhesion to wet surfaces, adhesion or integration of two unlike materials, and poor adhesive strength. Conventional bio-adhesives cannot account for one or more of these technical problems, rendering these bio-adhesives unsuitable for extended or long-term patient use.

Accordingly, there is a need for improved alternatives to conventional suturing.

### SUMMARY

In at least one aspect, a micro-structured mechanical adhesive that can be used in place of suturing is provided. The micro-structured mechanical adhesive includes a base section with a first side and a second side and a plurality of microstructures extending from the first side of the base section. Each microstructure is configured to yield in a predetermined direction upon adhesion to biological tissue and to provide attachment to biological tissue. Advantageously, the micro-structured mechanical adhesive is configured to provide attachment to biological tissue.

In another aspect, each microstructure includes a notch that biases the microstructure to yield, or buckle, in a predetermined direction and at a predetermined location along the structure.

In another aspect, the micro-structured mechanical adhesive provides a mechanical connection to the fibrous tissue, intestines, blood vessels, or nerves.

In another aspect, the micro-structured mechanical adhesive provides a mechanical connection in a wet environment.

In another aspect, a “sutureless” technology, MANTIS (Mechanical Adhesion to Tissue, adheres rapidly, is compliant yet strong to withstand dynamic conditions within the body, and is deployable in wet environments such as internal organs and wounds.

In another aspect, a growth-adaptive stent for use in a pediatric heart valve system is provided. The growth-adaptive stent includes a stent body defined by a plurality of struts and a plurality of spring elements. The plurality of struts provide structural support while the spring elements are configured to flex and expand between adjacent struts. The stent body can be composed of a superelastic material capable of transitioning between a compressed state and an expanded state and is configured to expand gradually in response to the growth of surrounding tissue when the growth-adaptive stent is implanted in a subject, thereby allowing the growth-adaptive stent to accommodate patient growth over time and wherein the spring elements exert a low chronic outward force, balanced by the surrounding tissue to control a rate of expansion.

In another aspect, a growth-adaptive heart valve prosthetic device is provided. The growth-adaptive heart valve prosthetic device includes a valve body configured to be implanted in a heart. Characteristically, the valve body has valve leaflets for regulating blood flow. Moreover, the valve body is composed of naturally occurring venous valve tissue. A growth-adaptive stent is attached to the valve body. The growth-adaptive stent includes a plurality of spring elements attached to a plurality of struts that provide structural support. Advantageously, the spring elements are configured to flex and expand, allowing the growth-adaptive stent to adapt to the growth of a patient's heart.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a further understanding of the nature, objects, and advantages of the present disclosure, reference should be had to the following detailed description, read in conjunction with the following drawings, wherein like reference numerals denote like elements and wherein:

FIG. 1A illustrates a schematic side view of a micro-structured mechanical adhesive.

FIG. 1B illustrates a perspective view of a two-dimensional array of hooks in a micro-structured mechanical adhesive undergoing preferentially yield or buckle in a predetermined direction.

FIG. 1C illustrates a schematic side view of a hook in a micro-structured mechanical adhesive undergoing failure in a predetermined direction.

FIG. 2 illustrates a schematic flowchart of an assembled micro-structured mechanical adhesive.

FIG. 3A illustrates a perspective view of a growth-adaptive stent.

FIG. 3B illustrates a side view of the growth-adaptive stent of FIG. 3A.

FIG. 3C illustrates schematics of a strut attachment to a micro-structured mechanical adhesive in a growth-adaptive stent.

FIG. 4A illustrates a cross-sectional view of a growth-adaptive heart valve device with a micro-structured mechanical adhesive.

FIG. 4B illustrates a top view of a growth-adaptive heart valve device with suturing.

FIG. 4C illustrates a cross-sectional view of a growth-adaptive heart valve device.

FIG. 4D illustrates a perspective view of a growth-adaptive heart valve device attached to an expandable stent.

FIG. 4E illustrates a perspective view of a growth-adaptive heart valve device attached to an expandable stent with an attached skirt.

FIG. 5A provides photographs of ancient weaponry and a praying mantis, which provide motivation for the micro-structured mechanical adhesive.

FIG. 5B provides brightfield micrographs of stainless steel microstructures used in the micro-structured mechanical adhesives.

FIG. 6 illustrates an example plot showing tissue thickness vs. proportion of observed vein wall thicknesses.

FIG. 7A illustrates an example of a testing system setup for testing single Claw and Jointed Claw microstructures using a glass block.

FIG. 7B illustrates an example plot showing distance vs. force for each Claw and Jointed Claw microstructure.

FIG. 7C illustrates representative images of key points of the force-position outlined in FIG. 7B.

FIG. 7D illustrates schematic views of controlled deformation for the Claw and Jointed Claw microstructures.

FIG. 7E illustrates an example chart for a yield force for the Jointed Claw and Claw microstructures.

FIG. 8A illustrates an example testing system setup for testing of single Claw and Jointed Claw microstructures using a tissue block.

FIG. 8B illustrates an example plot showing distance vs. force for each puncture and pullout microstructure.

FIG. 8C illustrates representative images of key points of the force-position outlined in FIG. 8B.

FIG. 8D illustrates an example chart for a yield force for the puncture and pullout microstructures.

FIG. 9A illustrates an example of a shear adhesion testing system setup for testing microstructures.

FIG. 9B illustrates an example plot showing distance vs. force for a shear testing system of the microstructures.

FIG. 9C illustrates representative images of key points of the force-position outlined in FIG. 8B.

FIG. 9D illustrates representative images of the microstructure array pre-test and post-test, showing entrapped tissue material.

FIG. 9E illustrates an example chart for a yield force for a shear testing system of the microstructures.

FIG. 10A illustrates an example peel adhesion testing system setup for testing microstructures.

FIG. 10B illustrates an example plot showing distance vs. force for a peel testing system of the microstructures.

FIG. 10C illustrates representative images of key points of the force-position outlined in FIG. 8B.

FIG. 10D illustrates an example chart for a yield force for a peel testing system of the microstructures.

FIG. 11A illustrates a block diagram of an example pediatric heart valve stent.

FIG. 11B illustrates an example stress simulation for normal and shear components.

FIG. 11C illustrates an example of stress simulation and force distribution along a single strut.

FIG. 11D illustrates an example stress simulation for normal and shear loads and an example acceptance matrix.

### DETAILED DESCRIPTION

Reference will now be made in detail to presently preferred compositions, embodiments, and methods of the present invention, constituting the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only to describe particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The phrase “composed of” means “including,” “comprising,” or “having.” Typically, this phrase is used to denote that an object is formed from a material.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

In the specific examples set forth herein, concentrations, temperature, and reaction conditions can be practiced with plus or minus 50 percent of the values indicated rounded to three significant figures. In a refinement, concentrations, temperature, and reaction conditions can be practiced with plus or minus 30 percent of the values indicated rounded to three significant figures of the value provided in the examples. In another refinement, concentrations, temperature, and reaction conditions can be practiced with plus or minus 10 percent of the values indicated rounded to three significant figures of the value provided in the examples.

### Abbreviations

“ANSYS” means a simulation software used for engineering analysis.
“BHV” means bioprosthetic valve.
“CAD” means computer-aided design.
“CFD” means computational fluid dynamics.
“cm” means centimeter.
“cP” means centipoise.
“FSI” means fluid-structure interaction.
“g” means gram.
“HFV” means human femoral vein.
“kg” means kilogram.
“LEAP” means low-force expanding/adaptable pediatric.
“m” means meter.
“MANTIS” means mechanical adhesion to tissue.
“mN” means millinewton.
“mm” means millimeter.
“N” means Newton.

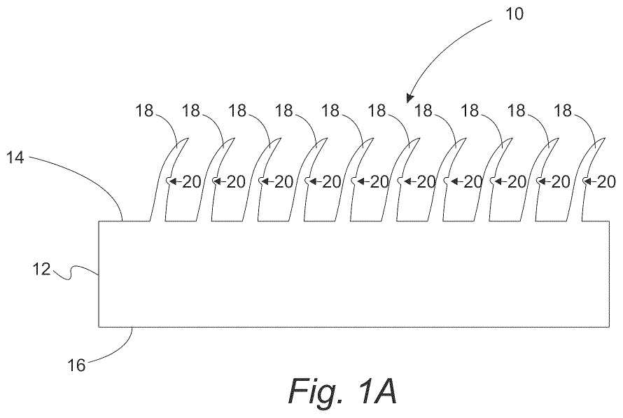

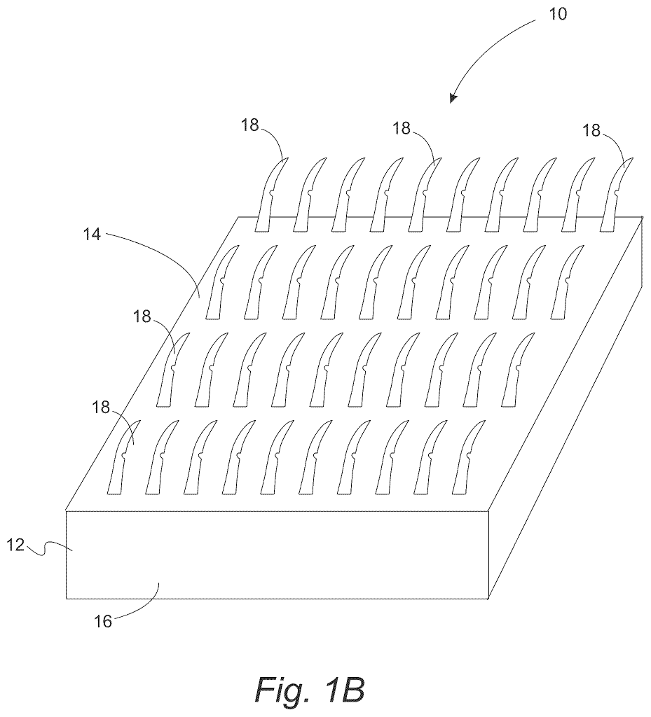

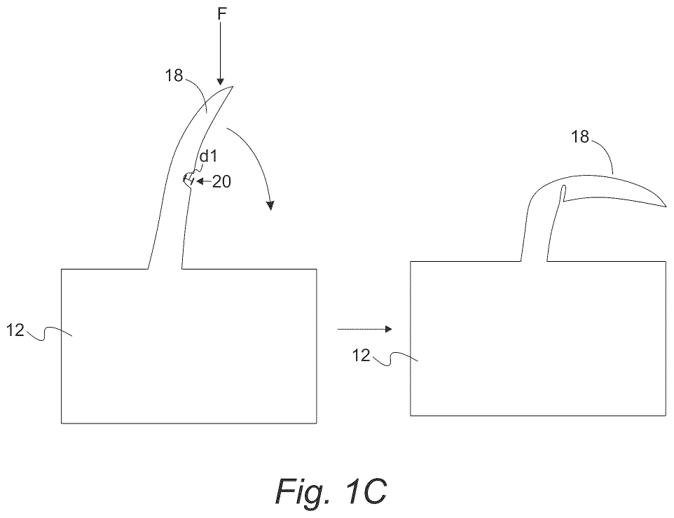

Referring to FIGS. 1A and 1B, a micro-structured mechanical adhesive (referred to as MANTIS) is schematically illustrated. Micro-structured mechanical adhesive 10 includes a base section 12 having a first side 14 and an opposite second side 16. A plurality of microstructures 18 extends from the first side 14 of the base section 12. Characteristically, each microstructure 18 is configured to preferentially yield or buckle in a predetermined direction. In a refinement, each microstructure 18 includes a hook that defines a preferential failure notch (i.e., a preferential yield point) that biases the hook to yield in the predetermined direction. The preferential failure notch defines a point of structural failure to bias the hook to yield in the predetermined direction by a threshold force. Advantageously, the preferential yield point can be located at any point along the microstructure and, in some embodiments, may manifest as a predefined leaning of the microstructure in the preferred direction. Examples of microstructures include hooks, pillars, and posts. Advantageously, the micro-structured mechanical adhesive is configured to provide attachment to biological tissue, thereby replacing conventional suturing.

In another aspect, the plurality of microstructures 18 may form hooks (referred to as fixed bend hooks) that curve as they extend from the first side 14 of the base section 12. In a refinement, each hook tapers as it extends from the first side 14 of the base section 12. In other words, the width of each hook narrows as it extends from the first side 14 of the base section 12. In a further refinement, each microstructure 18 includes barbs extending therefrom.

In another aspect, each microstructure 18 is characterized by a yield force that is adjustable by varying a notch depth d1, as labeled in FIG. 1C. In a variation, each microstructure 18 is characterized by a yield force that is adjustable by varying length proportions of sections below and above the notch. Different lengths lead to different yield forces for the same geometry since the moment applied at the notch is what produces material yield. Different lengths may also change tissue interaction and interlocking geometry.

In another aspect, the base section 12 and the plurality of microstructures 18 are unitary. In other words, the base section 12 and the plurality of microstructures 18 are made from the same material and are continuous with each other. In a refinement, the base section 12 and the plurality of microstructures 18 are composed of a metal. For example, the base section and the plurality of microstructures 18 are composed of nitinol or stainless steel. In a refinement, the base section 12 and the plurality of microstructures 18 are composed of a polymer.

In another aspect, the base section 12 and the plurality of microstructures 18 are non-unitary being composed of different materials. For example, the plurality of microstructures 18 can be an array of metal structures on a polymer base section 12.

Referring to FIG. 1C, the preferential failure notch 20 provides a preferred point of structural failure (i.e., by bending) when bending forces surpass a sufficient threshold defined as the yield force. Advantageously, notch 20 is configured to reduce the bending yield force. In a refinement, the bending yield force is less than 25 mN. Advantageously, bending of the microstructures can provide exceptional adhesive performance by promoting tight mechanical integration with the tissue.

In another aspect, each microstructure 18 has a length of about 50 to 250 microns. In another aspect, each microstructure 18 has a base proximal to the base section, where the width at the base is from 10 to 50 microns.

In another aspect, the plurality of microstructures is arranged as a one-dimensional array, as depicted in FIG. 1A. Alternatively, the plurality of microstructures can be arranged as a two-dimensional array, as depicted in FIG. 1B.

In another embodiment, a method of suturing with the micro-structured mechanical adhesive set forth above is provided. The method includes a step of attaching the micro-structured mechanical adhesive to a biological tissue such that the plurality of microstructures extend into the biological tissue to form a mechanical connection. In a refinement, the biological tissue is a fibrous tissue, intestines, blood vessels, or nerves. Advantageously, the micro-structured mechanical adhesive can be attached to a medical device (e.g., a pediatric stent).

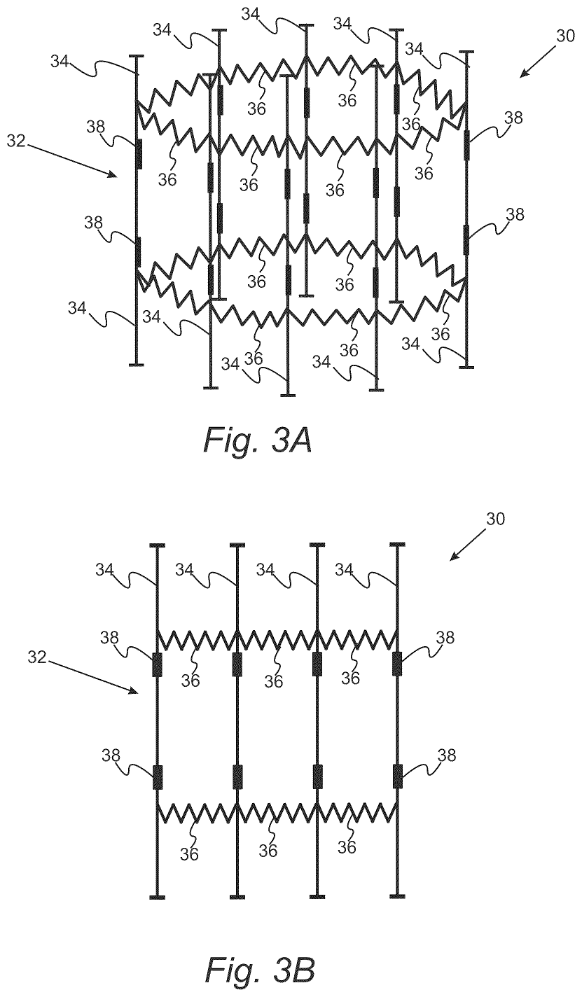

In another aspect, the micro-structured mechanical adhesives described above can be fabricated using various methods. For example, the adhesive can be produced through laser machining or micro-casting. Alternatively, metal substrates can be patterned using photoresists and then chemically etched. The preferentially bending structures mentioned here are a type of microstructure that has shown highly promising results in early feasibility testing. These structures are made from metallic sheets, such as stainless steel or nitinol. They are specifically designed to pierce into, but not fully puncture, thin tissue and then bend in a controlled manner to securely attach the structure to the tissue. This is accomplished through careful design of the microstructures, precision etching during fabrication, and specific handling and assembly processes. While the designs may vary based on the application, they typically include a notched section that causes the microstructure to bend or buckle at that point, allowing precise control over its behavior. Examples of applications for the microstructured adhesive include medical devices (such as the stent depicted in FIG. 3), anastomosis procedures, wound closure, skin adhesion, hemorrhage control, and other related uses. Additionally, the microstructured adhesive has potential for various non-biological applications, such as bonding materials in microelectronics, securing components in aerospace technology, providing mechanical adhesion in robotics, and even enhancing adhesion in construction materials for structural integrity.

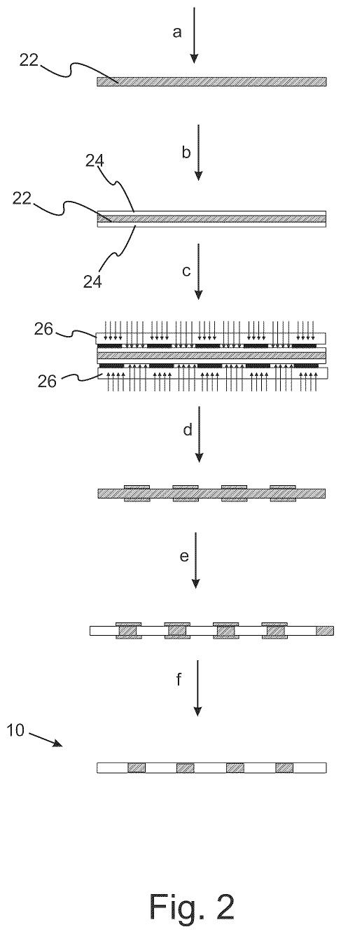

Referring to FIG. 2, a method for forming micro-structured mechanical adhesive 10 is provided via a schematic flowchart. In step a), the foil 22 is cleaned. In step b) a coating 24 of photoresist is applied to both faces. A photo tool containing the designs is then used as a mask upon exposure to UV light. In step c), the photoresist coating 24 is exposed to UV light through a mask 26. In step d), the photoresist is developed. In step e), metal unprotected by the photomask is etched away. In step f), the photoresist is stripped, leaving the stainless steel microstructure arrays (i.e., micro-structured mechanical adhesive 10).

In another aspect, a growth-adaptive stent for use in prosthetic devices such as a pediatric heart valve system is provided. In some cases, one or more components of the growth-adaptive stent can include the mechanical adhesives described herein. Referring to FIGS. 3A, 3B, and 3C, growth-adaptive stent 30 includes a stent body 32 defined by a plurality of struts 34 and spring elements 36. Struts 34 provide the necessary structural support, while the spring elements 36 are configured to flex and expand. In a refinement, the stent body 32 (i.e., the struts and/or the spring elements) is composed of a superelastic material, such as nitinol, which is capable of transitioning between a compressed state and an expanded state. Moreover, the growth-adaptive stent 30, which is composed of a superelastic material, provides a low chronic outward force that allows for controlled expansion in response to the growth of surrounding tissue. In a refinement, the struts 34 and spring elements 36 may be coated with a biocompatible material to reduce tissue ingrowth and prevent inflammatory responses, ensuring long-term safety and functionality within the body. Additionally, other growth-adaptive stents or medical devices are possible, including those designed to expand or adapt in alternative ways to accommodate different biological conditions and patient needs.”

In another aspect, stent body 32 is further designed to maintain its structural integrity and functionality across various diameters, from a first compressed diameter to a second expanded diameter. Specifically, the stent is configured to expand from an initial diameter of about 8 mm to a second diameter of about 16 mm, accommodating the growth of the heart in pediatric patients.

In another aspect, the spring elements 36 are designed to enable controlled, non-invasive expansion, preventing immediate full expansion upon implantation and instead enabling gradual adaptation as the patient grows. This ensures that the expansion of the growth-adaptive stent 30 occurs in a controlled manner, allowing the stent to adjust gradually as needed without causing undue stress on the surrounding tissue. Once the stent 30 reaches its maximum expanded diameter, the sent 30 is configured to reach a zero-stress state, after which no further active expansion occurs. This ensures that the stent remains stable once the desired diameter is achieved, preventing unnecessary forces from being exerted on the tissue.

In another aspect, growth-adaptive stent 30 can also include sections of micro-structured mechanical adhesive 10 attached to struts 34, as described above. In a refinement, microwelding features along struts 34 allow for the attachment of valve components using mechanical adhesion technology. This enables a sutureless integration of the valve into the stent 30, simplifying the implantation process while maintaining a secure connection between the valve and the stent body. FIG. 3C shows a variation where strut 34 includes slot features 38 for attaching a section of micro-structured mechanical adhesive 10.

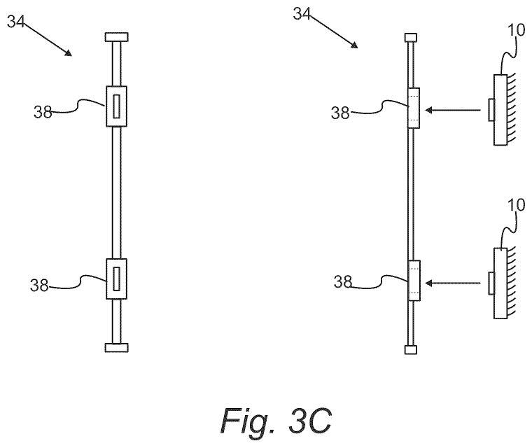

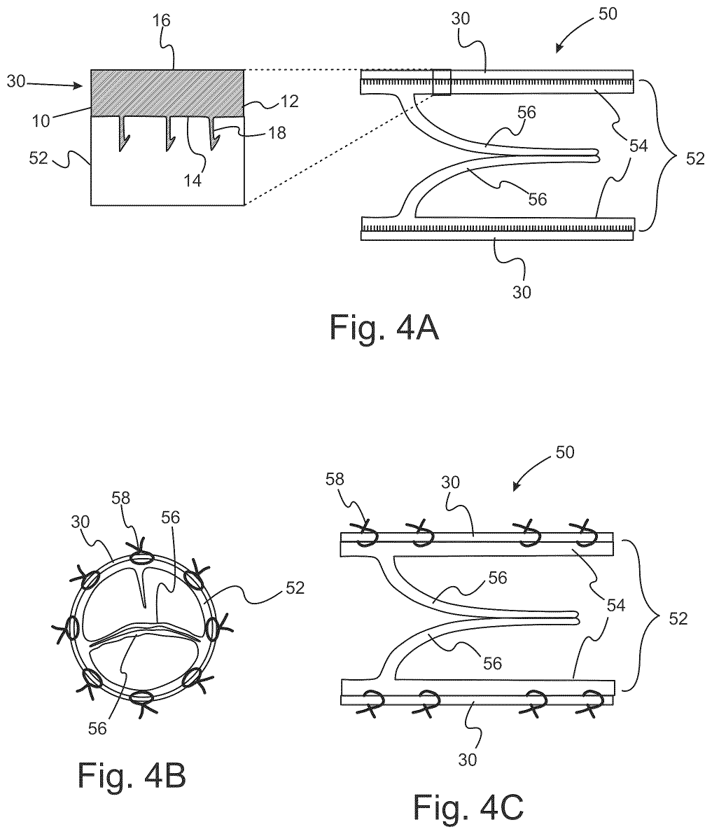

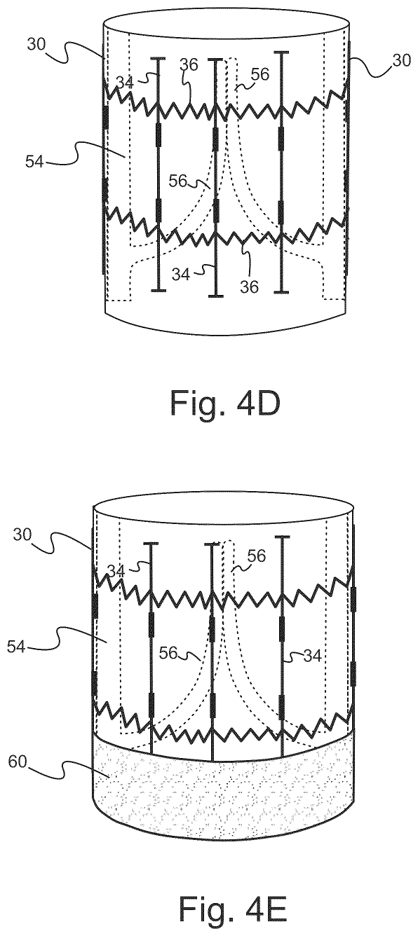

Referring to FIGS. 4A, 4B, 4C, and 4D, schematics of a growth-adaptive heart valve device are provided. Growth-adaptive heart valve device 50 is a Low-force Expanding/Adaptable Pediatric (LEAP) Valve. Growth-adaptive heart valve device 50 includes a valve body 52 configured to be implanted in a heart. The valve body 52 includes an outer tubular section 54 and a plurality of leaflets 56 attached thereto for regulating blood flow. Advantageously, the valve leaflets 56 are configured to operate across at least a two-fold diameter range, allowing the valve to accommodate growth from infancy through early childhood. In a refinement, the valve body 52 is composed of naturally occurring venous valve tissue (e.g., human femoral vein). The device further includes a stent 30 comprising a plurality of spring elements 36 attached to a plurality of struts 34 that provide structural support, as described above. The spring elements 36 are configured to flex and expand, allowing the stent 30 to adapt to the growth of the patient's heart while maintaining low radial force.

In another aspect, growth-adaptive stent 30 is attached to the valve body 52. In a variation as depicted in FIGS. 4B and 4C, stent 30 is sutured to valve body 52 with sutures 58. In another variation, the micro-structured mechanical adhesive is micro-welded to struts 34, as described above. In a refinement, the growth-adaptive stent 30 is attached to the valve body with the micro-structured mechanical adhesive configured to attach the growth-adaptive stent 30 to cardiac tissue. Typically, the micro-structured mechanical adhesive includes re-entrant microstructure geometries forming a mechanical interlock between the valve body and the growth-adaptive stent 30, designed to minimize local stress concentrations and reduce tissue tearing. As previously described, the micro-structured mechanical adhesive 10 includes a base section 12 having a first side 14 and a second side 16, with a plurality of microstructures 18 extending from the first side of the base section 12 as depicted in FIG. 1A. Each microstructure 18 is configured to yield or buckle in a predetermined direction preferentially. The valve body 52 and the stent 30 are further configured to provide continuous valve functionality over a predetermined expansion range, maintaining proper leaflet motion and blood flow regulation at all diameters. The micro-welding aspect of the growth-adaptive heart valve device 50 focuses on integrating microstructures with the stent 30 through a precise and controlled welding process. Micro-welding is used to attach the microstructure arrays to the longitudinal struts of the stent 30, ensuring a secure and reliable connection. This technique allows for the creation of a strong bond between the nitinol stent and the microstructures, which are designed to improve valve performance by distributing loads evenly and enhancing adhesion without the need for sutures. Micro-welding is particularly critical due to the small size of the components, as the growth-adaptive heart valve device 50 is specifically designed for pediatric use. The process allows for a sutureless assembly, reducing time, variability, and skill requirements in the manufacturing process, and is a key innovation in the valve's design.

In another aspect, growth-adaptive heart valve device 50 includes a polymer skirt 60. The polymer skirt is a useful feature designed to improve the performance and longevity of the growth-adaptive heart valve device 50 by acting as a physical barrier between the device and host tissue. Its primary function is to reduce pannus tissue ingrowth, a common issue in rapidly growing children, which can lead to valve dysfunction. The polymer skirt 60 is designed to minimize tissue reaction and inflammation by preventing direct contact between the valve and surrounding tissues. In a refinement, polymer skirt 60 is composed of thin, biocompatible materials such as ePTFE or thermoplastic polyurethanes, chosen for their inert properties and existing regulatory approval as stent coatings. Polymer skirt 60 is designed not to interfere with the valve's expansion or function, with options for either full or partial coverage of the stent 30 and continuous or interrupted designs to allow for growth adaptation. Polymer skirt 60 can be evaluated through in vitro and in vivo studies, with radial force testing and hydrodynamic performance assessments to ensure it enhances valve function without compromising its growth-adaptive capabilities.

In another aspect, the adventitial layer in the growth-adaptive heart valve device 50 is modified to improve valve performance, particularly at smaller diameters, by reducing the thickness of the human femoral vein (HFV) graft's outermost layer. This is essential for pediatric patients, especially neonates, and infants, where the valve must function efficiently at diameters under 10 mm. Excess tissue in the adventitial layer can reduce valve performance, increase pressure gradients, and trigger immune responses. To address this, the layer is trimmed or mechanically stripped to achieve a target thickness of 200-250 μm. This increases the valve's effective orifice area, improves blood flow, reduces complications, and ensures better integration with the stent 30. The process involves pressurizing the graft, identifying excess tissue, and carefully removing the outer layer, optimizing valve function in young patients.

The following examples illustrate the various embodiments of the present invention. Those skilled in the art will recognize many variations that are within the spirit of the present invention and scope of the claims.

### 1. Metallic Microstructured Fasteners For Mechanical Adhesion To Soft Tissues

### 1.1 Overview

In the following experiments, the mechanical adhesion of microstructures (i.e., MANTIS) to tissue was evaluated. The microstructures are a probabilistic fastening system composed of re-entrant metal microstructures that enable rapid and reliable attachment between biocompatible metals and soft tissue. The system aims to address the challenges of suturing during the assembly of pediatric bioprosthetic heart valves (BHV), where each suture must be carefully positioned around stent structures to avoid damaging valve function (FIG. 4B). The microstructure is a microstructured mechanical adhesive, which would allow for a faster and more reliable method of attaching the valve to the stent (FIG. 4A). To inform the design of microstructures, the wall thickness of human femoral vein (HFV) graft tissue used in the pediatric BHV device was measured. Several microscale designs may be appreciated. These designs demonstrated rapid and robust tissue adhesion under normal, shear, and peel loading modes. Some of the more novel biomimetic designs included deformation behaviors that enhanced tissue entrapment during puncture. Finally, an in silico evaluation of adhesion performance under simulated worst-case loading conditions in vivo suggested that integrating microstructure with BHV devices is feasible. FIG. 4B highlights the challenges of suture-based BHV assembly, while FIG. 4A presents microstructure as the proposed solution.

### 1.2 Materials And Methods

### 1.2.1 Human Femoral Vein Tissue Characterization

### 1.2.1.1 Histology

Human Femoral Vein (HFV) grafts (CRYOLIFE®) were stored at −80° C. before thawing for histological preparation. Once thawed, HFVs were kept hydrated with 1× phosphate-buffered saline (PBS) unless specified otherwise. The HFV was inspected to locate venous valves, whose positions were tracked through the subsequent sectioning procedure.

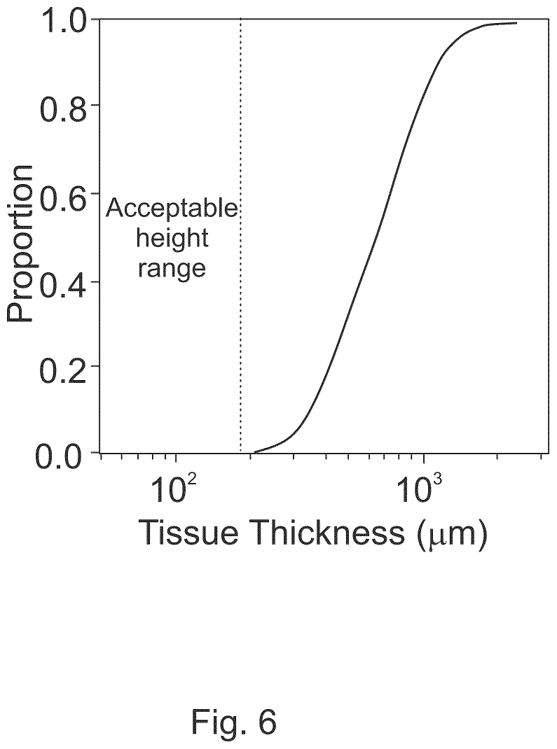

FIG. 6 provides an empirical cumulative distribution function for observed vein wall thickness with the acceptable microstructure height to avoid fully puncturing through the valve graft wall indicated. Custom-fabricated polydimethylsiloxane (PDMS) end caps with filling ports were sutured onto each end of the HFV segment. The HFV was drained of PBS, then slowly filled with optimal cutting temperature (OCT) compound (Tissue-Tek) using a syringe. Once filled, the HFV was disconnected from the syringe and transferred into a 50 mL conical tube, which was then filled with OCT to submerge the HFV fully. After freezing at −80° C., the embedded HFV was removed from the conical tube and cut into ˜5 mm thick subsections using a jeweler's saw. Subsections were placed in cryomolds (25× 20×5 mm cryomold, Andwin Scientific), snap-frozen in liquid nitrogen, and then underwent cryo-sectioning and histological staining (The Koch Institute, Swanson Biotechnology Center, MIT). Masson's trichrome stain was used on 5-10 μm thick sections to visualize extracellular matrix proteins within the HFV wall using brightfield microscopy.

### 1.2.1.2 Measurements of Human Femoral Vein Wall Thickness

Separate HFV samples were prepared for thickness measurements using a custom preparation and sectioning method. Frozen HFV samples were thawed, hydrated in 1×PBS, and then drained. HFVs were filled with and submerged into a 10:1 mixture of liquid PDMS and crosslinking agent using a 3D-printed mold. The PDMS was then cured at 65° C. for 60 min. The encased HFV was removed from the mold and sectioned using a razor blade. Brightfield microscopy images of sections were acquired for wall thickness measurement. Images were then loaded into an image analysis software for measurement (FIJI). HFV wall regions were segmented from the valve and background regions using a user-set threshold, producing a binarized mask. A polar transformation was then applied to the masks such that the HFV wall region appeared rectangular to improve ease of measurement. The thickness of the wall region was measured at 0.25° intervals using the polar transformed masks, and thickness data were subsequently exported for statistical analysis.

### 1.2.2 Design and Fabrication of Metal Microstructures

The microstructures were fabricated by a commercial vendor (Resonetics, LLC) using a photochemical machining (PCM) process (FIG. 2). Briefly, a photo tool was created from the microstructure geometries designed with CAD software (SolidWorks 2017, Dassault Systèmes SE). One-dimensional arrays of 42 microstructures were incorporated on the edge of a 1 cm×0.5 cm coupon of 0.001″ (˜25 μm) thick 302 full hardness stainless steel foil. After the foil was cleaned, a photoresist coating was applied to both faces. The photo tool containing the designs was then used as a mask upon exposure to UV light. Next, the photoresist was developed, exposing the regions of metal to be removed, which was subsequently chemically etched. The remaining photoresist was stripped, leaving the stainless steel microstructure arrays.

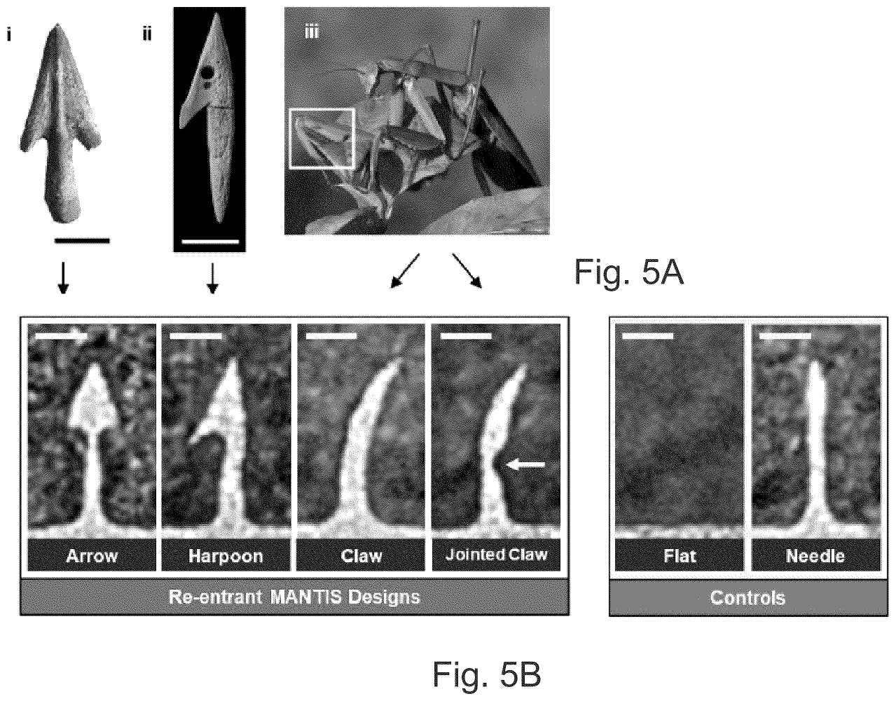

Re-entrant designs included the “Arrow,” “Harpoon,” “Claw,” and “Jointed Claw” (FIG. 5). Two controls were also fabricated: a “Flat” edge to control for adhesive contributions from the base region around the microstructures and a “Needle” design that controls for the presence of re-entrant features.

### 1.2.3 Mechanical Testing of Single Microstructures

### 1.2.3.1 Normal Orientation Testing System

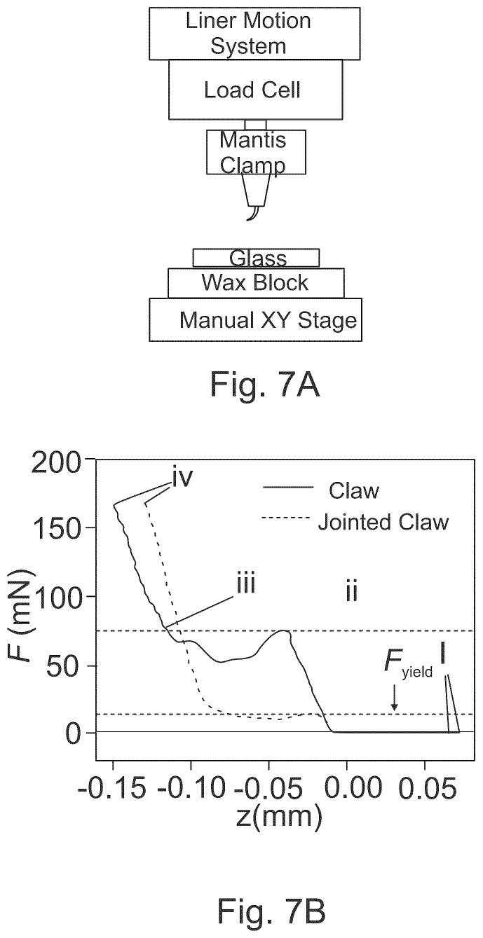

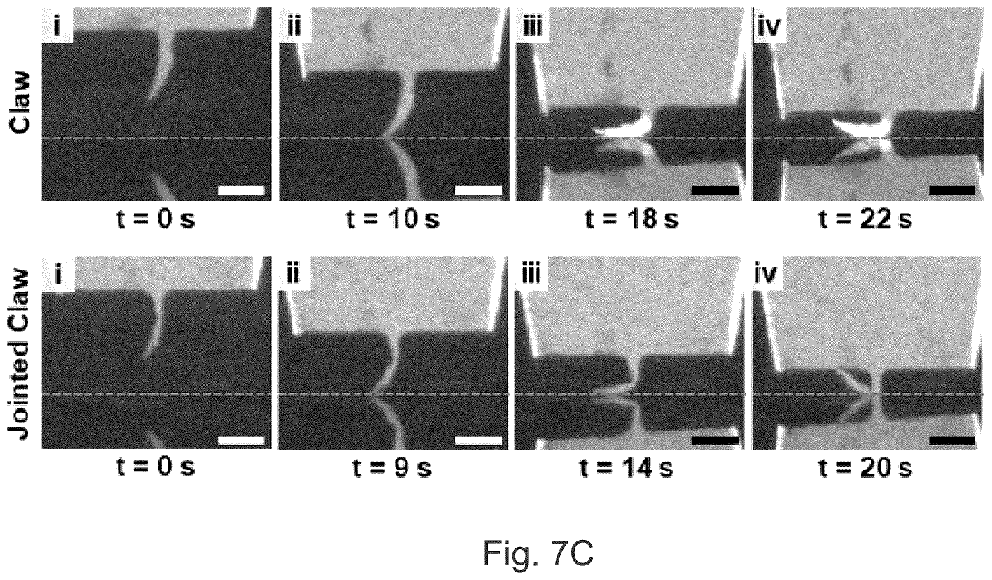

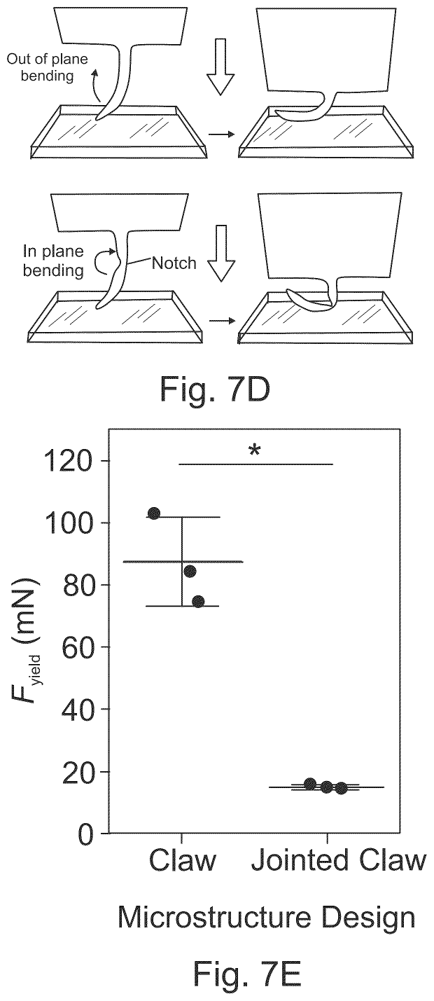

Bending yield and tissue pullout testing of individual microstructures was conducted using a custom-built uniaxial loading measurement instrument (FIG. 7A). Force measurements were taken with a high-sensitivity load cell (LFS 242.25, Group Four Transducers) and amplified with a digital load cell amplifier (DAD 141.1, Flintec, Inc.), providing a resolution of 1 mN. Data acquisition and motion control were coordinated through a DAQ (USB-6210, National Instruments) and a custom LabVIEW virtual instrument (LabVIEW 2021, National Instruments). Normal force measurements (FN) were collected at a sampling rate of 10 Hz, while images were simultaneously captured at a rate of 1 Hz using a USB microscope (Edge AM73915MZTL, Dino-Lite).

### 1.2.3.2 Bending Yield Testing

Bending yield testing of individual Claw and Jointed Claw microstructures was performed using the uniaxial force measurement instrument and acquisition parameters described above in section 1.2.3.1. Alignment holes were laser cut into the main body of the coupon using a UV laser cutter (LPKF, ProtoLaser U4). Individual microstructure samples were prepared by manually trimming coupons with titanium-coated stainless steel micro-scissors (Micro-Mark, catalog #89589) to isolate a single microstructure at the center of the coupon. Microstructure samples were aligned and loaded into a custom 3D-printed sample clamp using laser-cut alignment holes, with the clamp secured to the load cell. The sample was then lowered onto a rigid glass substrate at a speed of 25 μm/s until the microstructure reached complete failure. Bending yield experiments were conducted in triplicate.

FIG. 7 describes bending yield testing of single-claw and jointed-claw microstructures. FIG. 7A illustrates the custom testing system setup with components labeled. FIG. 7B shows a representative force-position graph for both Claw and Jointed Claw microstructures during bending yield tests. Key points on the graph are highlighted with corresponding images in FIG. 7C, including (i) the approach, (ii) the initiation of yield, (iii) the end of the plateau, and (iv) the fully bent structure after the test. A dashed gray line marks the contact surface, and the scale bar represents 100 μm. FIG. 7D demonstrates the controlled deformation concept, where the Claw microstructure tends to bend out-of-plane when pressed against a rigid surface, while the Jointed Claw design, with its notched joint, provides more controlled bending motion. Finally, FIG. 7E shows that the yield force was significantly reduced in the Jointed Claw design compared to the Claw design. The mean values are indicated by lines, and error bars represent the standard deviation.

### 1.2.3.3 Normal Pullout of Single Microstructures

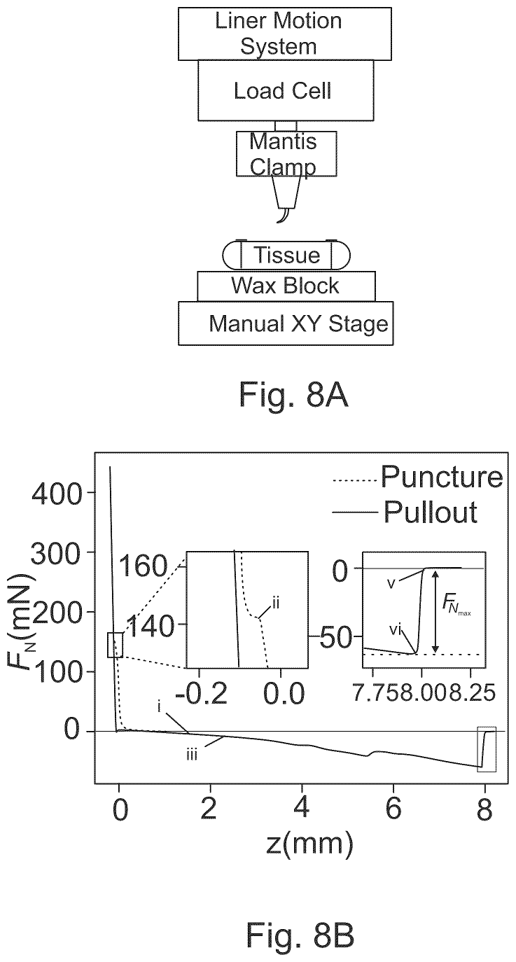

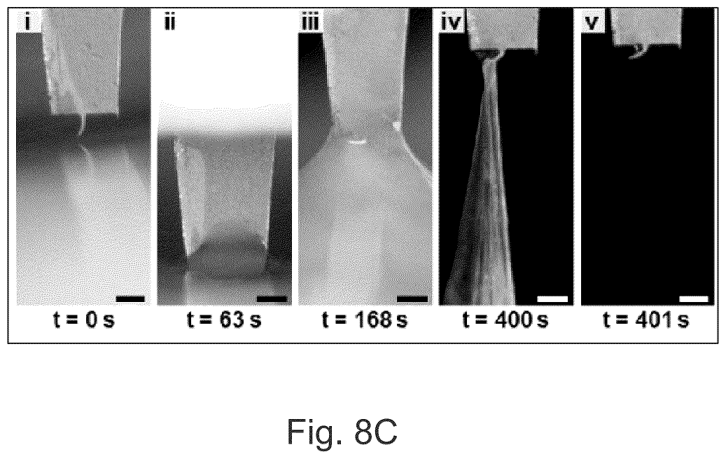

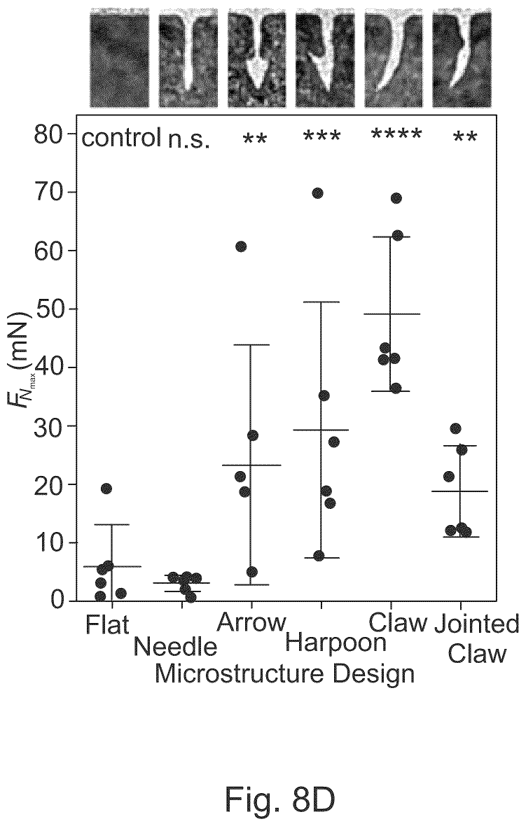

Pullout strength testing of individual MANTIS microstructures was performed using the same uniaxial force measurement instrument and acquisition parameters described earlier in section 1.2.3.1 (FIG. 8A). The microstructure samples were prepared according to the method detailed in section 1.2.3.2. A rectangular piece of HFV tissue (˜3×3 cm) was pinned at each corner to a wax block (Catalog #9389K51, McMaster-Carr), with the adventitial side facing up toward the microstructures. The tissue was positioned so that the vessel's longitudinal axis was parallel to the plane of the MANTIS sample. During testing, the tissue was kept hydrated by frequently applying 1× PBS. The test began by lowering a single microstructure into the tissue until a compressive force of 400 mN was reached, a value determined from pilot experiments. Motion was then paused for 5 seconds to allow the tissue to relax mechanically. Afterward, the microstructure was pulled upward until the mechanical connection between the microstructure and the tissue completely failed. A constant speed of 25 μm/s was maintained during both the puncture and pullout phases. All re-entrant designs were compared against the flat condition as a control, with the needle design, which lacks re-entrant features and serves as a negative control. The performance was quantified using the maximum tensile force (F_{Nmax}) measured during the test. Normal pullout experiments were conducted with a sample size of N=6. FIG. 8A illustrates the custom puncture testing system and its components, while FIG. 8B shows a representative force-position curve for the puncture and pullout of a single MANTIS microstructure. The key stages of this process are illustrated in FIG. 8C, including (i) the approach, (ii) a force dip due to microstructure bending or tissue puncture, (iii) the early stages of pullout, (iv) the maximum pullout force, and (v) complete removal of the microstructure from the tissue. The corresponding images are taken from different z-positions, with a scale bar of 200 μm. Finally, FIG. 8D shows the maximum pullout force for single microstructures, highlighting one outlier for the Flat design, where tissue adhesion to the support strip resulted in a higher pullout force. Mean values are represented by lines, and error bars indicate standard deviation.

### 1.2.4 Mechanical Testing of Microstructure Arrays

For shear and peel testing, all asymmetric designs (i.e., Harpoon, Claw, and Jointed Claw) were evaluated in both primary (i.e., the tip of the hook or barb feature facing away from the loading direction) and secondary (i.e., the tip of the hook or barb feature facing toward the loading direction) orientations. In addition to the re-entrant designs, three controls were evaluated: the structure-less Flat and non-re-entrant Needle, in addition to a condition consisting of “Empty” coupon clamps for measuring the adhesive contribution of the coupon clamp face.

### 1.2.4.1 Shear Orientation Testing System

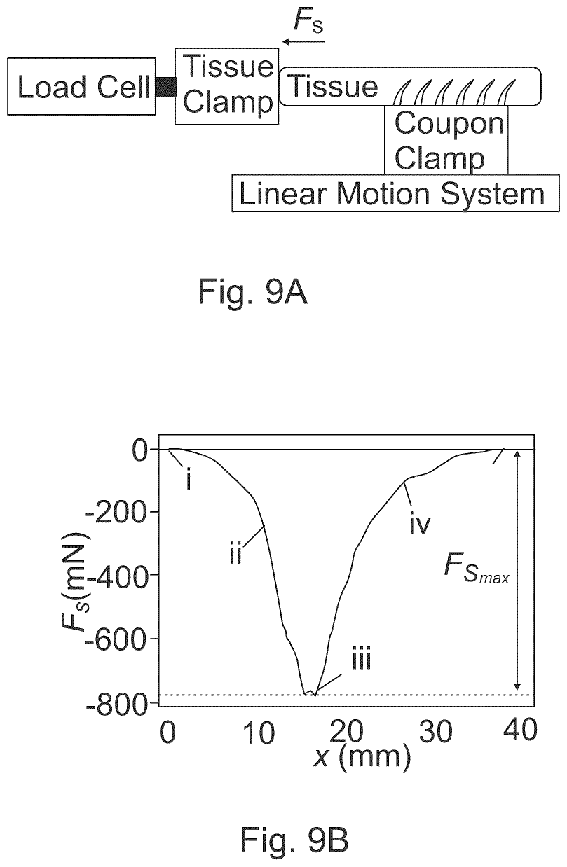

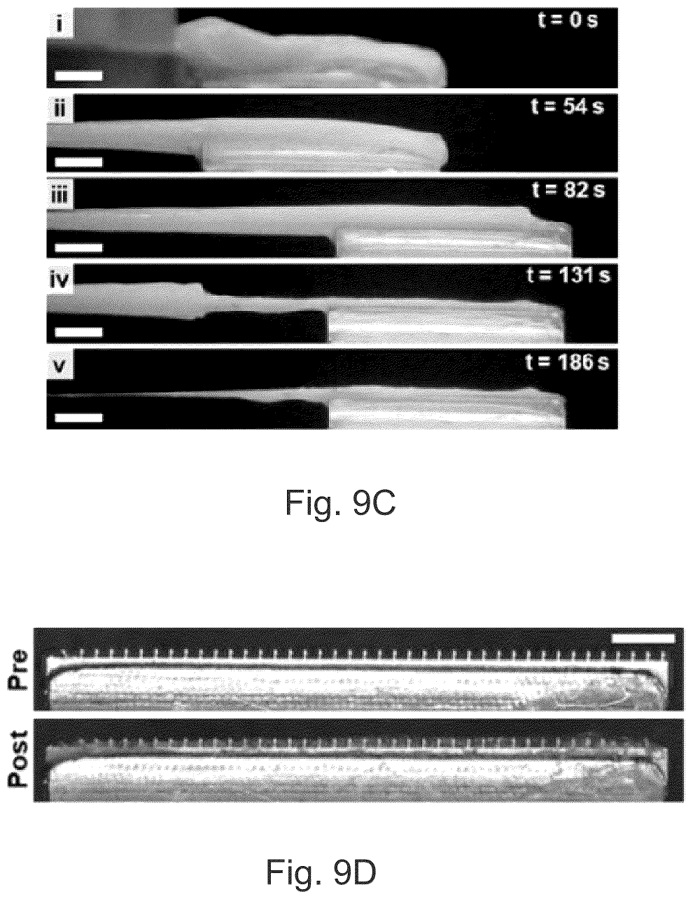

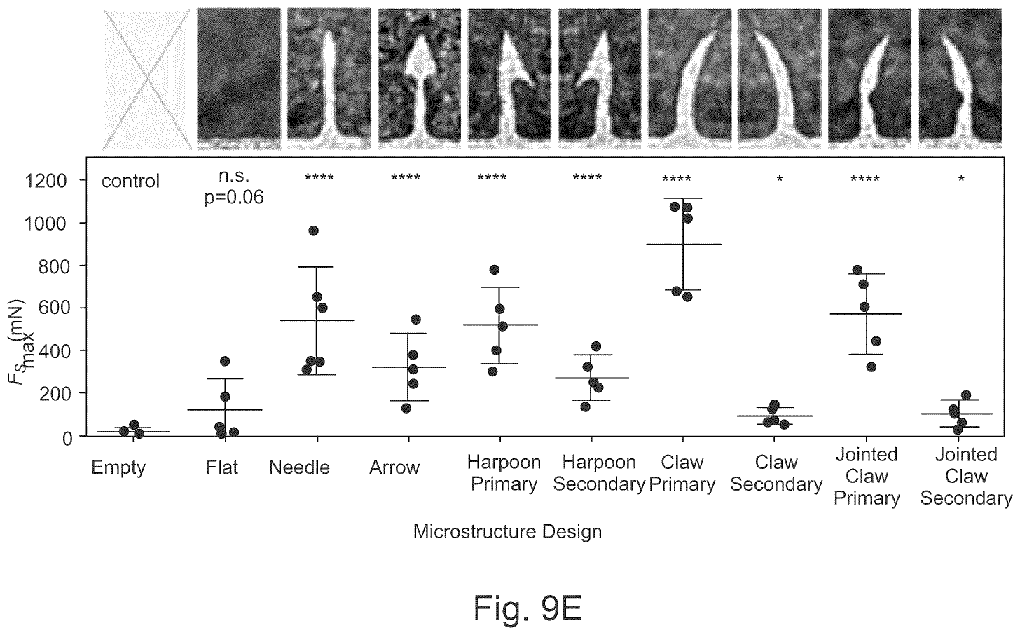

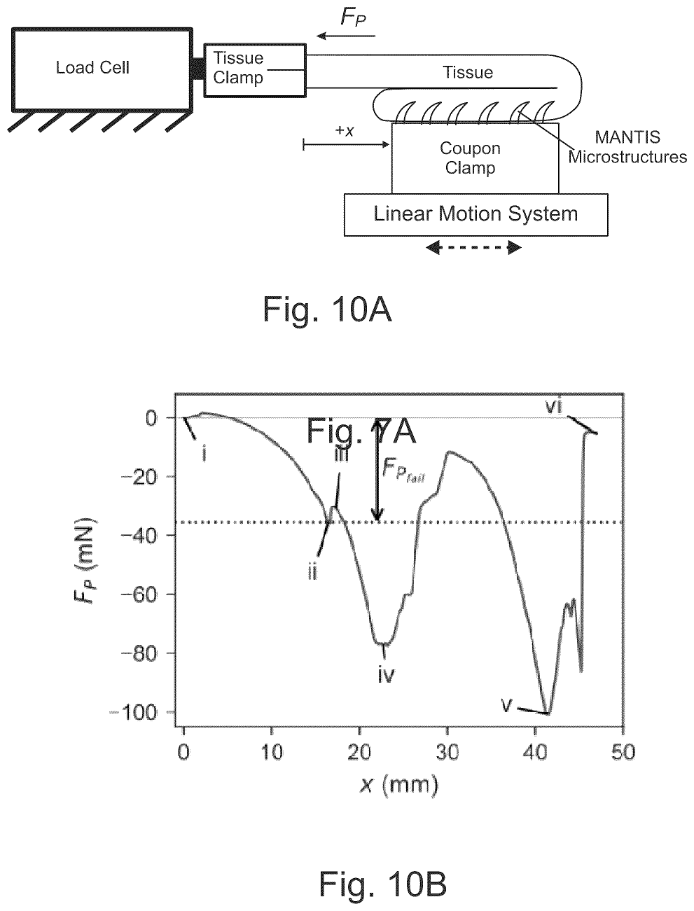

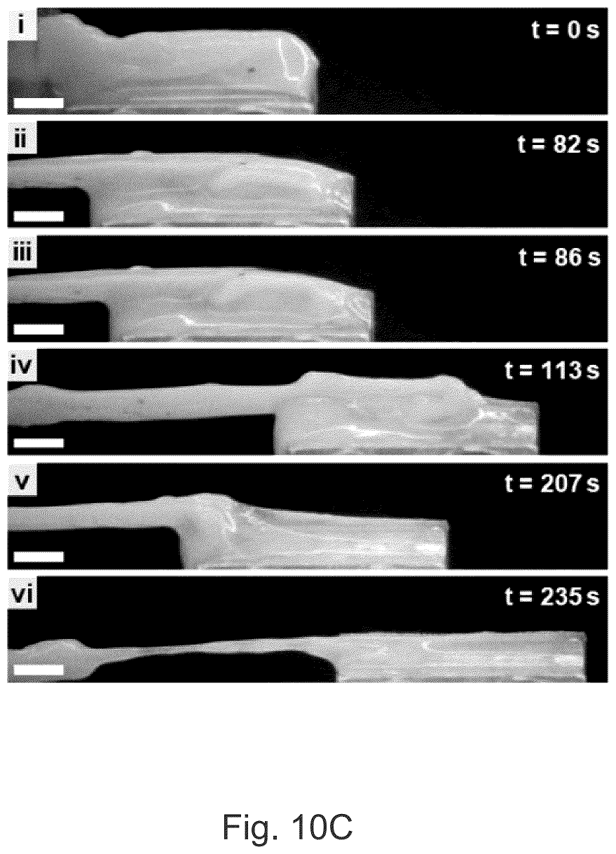

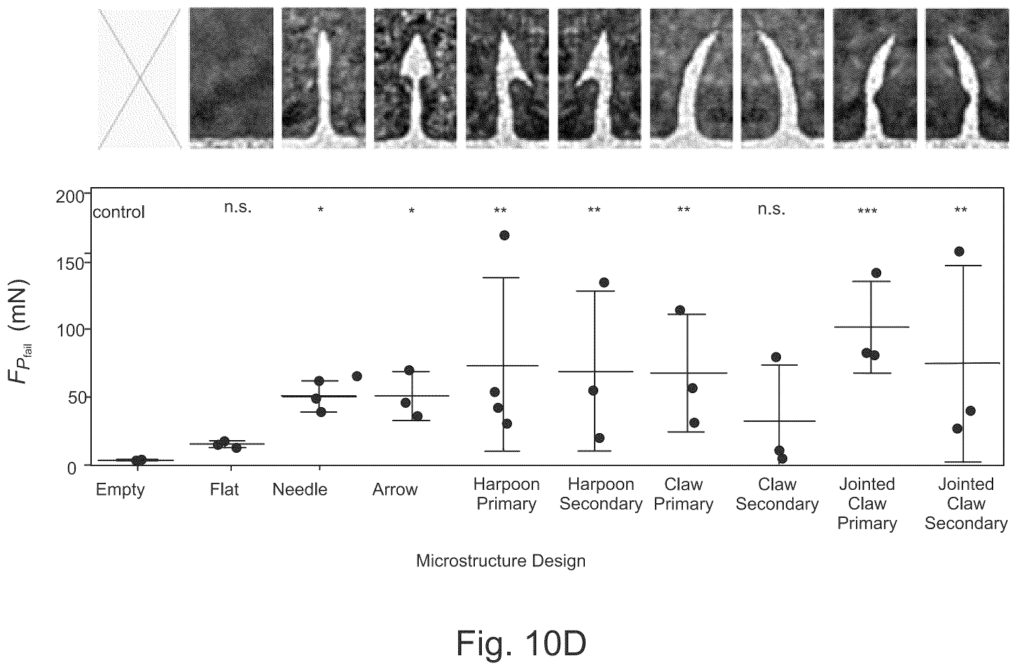

Shear and peel adhesion testing was conducted using a custom-built uniaxial measurement instrument (FIGS. 9A, 10A). MANTIS coupons were secured to a moving stage driven by a linear motion system using a custom-machined aluminum coupon clamp. The system employed the same load cell, load cell amplifier, USB microscope, control software, and data acquisition settings as those used for the normal orientation testing system described in section 1.2.3.1. The force during the tests was recorded as F_{S} for 0° shear testing and F_{P} for 180° peel testing. A custom 3D-printed tissue clamp was used to couple the load cell to the HFV tissue sample being evaluated.

FIG. 9 illustrates the setup for shear adhesion testing of MANTIS microstructure arrays. In FIG. 9A, a diagram of the custom shear testing system is shown with labeled components, where the adventitial surface of the tissue faces the microstructure array. FIG. 9B displays a representative force curve for a shear adhesion test of a MANTIS microstructure array, specifically a Harpoon array oriented in the primary direction. Key phases of the pullout process are captured in FIG. 9C, including (i) initiation, (ii) progressive tissue loading, (iii) maximum shear adhesion force coinciding with MANTIS adhesion failure, (iv) cohesive tissue failure, and (v) test completion. These images were taken from different x-positions, with a scale bar of 2 mm. FIG. 9D shows photographs of the MANTIS microstructure array before and after testing, highlighting the entrapped tissue material (scale bar=1 mm). Finally, FIG. 9E presents the maximum shear adhesion force for the arrayed microstructures, with lines representing mean values and error bars indicating standard deviation.

FIG. 10 illustrates the peel adhesion testing of MANTIS microstructure arrays. In FIG. 10A, the diagram shows the custom peel testing system with labeled components, where the adventitial surface of the tissue sample is positioned facing the microstructure array. FIG. 10B presents a representative force curve for the peel adhesion test of a MANTIS microstructure array. Key points on the graph are depicted in FIG. 10C, using images of the Arrow design, showing (i) the initial loading and stretching of the HFV tissue, (ii) the first detachment of the tissue from the microstructures, (iii) reloading of the microstructures, (iv) the local maximum adhesion force, (v) the global maximum adhesion force, and (vi) the final detachment of the tissue from the MANTIS microstructure array. These images were captured from different x positions, with a scale bar of 2 mm. FIG. 10D shows the initial failure force for the microstructure arrays, with mean values indicated by lines and error bars representing standard deviation.

### 1.2.4.2 0° Shear Pullout of Microstructure Arrays

Arrayed MANTIS microstructure samples were prepared for shear pullout testing by cutting alignment holes with a UV laser cutter (LPKF ProtoLaser U4). MANTIS coupons were then secured in the coupon clamp such that all microstructures were exposed above the clamp surface. Images of the clamped MANTIS coupons were captured before and after testing using an optical profilometer (VR-6000, Keyence). HFV tissue samples were cut into 2.5 cm×0.5 cm rectangular strips to provide sufficient length to reach between the tissue and coupon clamps. Tissue samples were gently laid on top of the coupon clamp with the adventitial side facing the microstructure array and oriented such that the longitudinal axis of the vessel was parallel with the plane of the MANTIS coupon. Once aligned, the tissue was attached to the microstructure array using an arbor press to briefly apply 1000 gf, which was determined to produce successful bonding in pilot tests. The microstructure-tissue assembly was mounted on the testing platform, and the height of the load cell was adjusted to align the loading axis with the plane of the tissue sample. The tissue was then stretched at a speed of 200 μm/s until failure of the microstructure-tissue connection. Performance was quantified via the metric F_{Smax}, defined as the maximum tensile force reached during the test. Shear test experiments were run with a sample size of N=5-6.

### 1.2.4.3 180° Peel of Microstructure Arrays

Peel testing was executed on the same testing system described in 1.2.4.1. Sample preparation was similar to the process described in 1.2.4.2, with the exception of HFV samples that were cut into 4 cm×0.5 cm rectangular strips before adhesion to MANTIS coupons via pressing. A 180° peel loading configuration was applied by folding the tissue back over itself before securing it to the tissue clamp (FIG. 10A). The tissue was then peeled at a speed of 200 μm/s until failure of the MANTIS-tissue connection. Performance was quantified via the metric F_{Pfail}, defined as the force at which the tissue began to separate from the microstructure array. Synchronous image data was manually compared with force-position curves to identify the first instance of adhesive failure, and F_{Pfail} was recorded as the associated local peak in tensile force. Peel experiments were run with a sample size of N=3-4.

### 1.2.5 in Silico Evaluation of MANTIS Feasibility for BHV Assembly Application

### 1.2.5.1 Bioprosthetic Heart Valve Model

A one-way fluid-structure interaction (FSI) model of a pediatric BHV device was created using Multiphysics simulation software (ANSYS Fluent and ANSYS Static Structural Analysis) to examine loads exerted by blood flow on the device assembly. The 3D model of the 15 mm long device was set within a cylinder of diameter 7 mm representing a neonatal pulmonary artery (PA) implantation site. Three simulation configurations were investigated: (1) diastole, (2) systole prohibiting paravalvular flow between the HFV and PA surfaces (systolic flush), and (3) systole allowing paravalvular flow between the HFV and PA surfaces (systolic gap). Simplified valve geometries were used, with valve leaflets absent in systolic configurations or represented as a flat wall in diastolic configurations. Steady-state computational fluid dynamics (CFD) blood flow simulations were performed assuming laminar flow with a dynamic viscosity of 3.5 cP and a density of 1060 kg/m3. Flow rate boundary conditions were used in systolic configurations, while pressure boundary conditions were used in diastolic configurations. Wall stresses from the CFD results were then applied to the structural model. The stent and PA walls were modeled as rigid bodies, and the HFV tissue was modeled as a linear elastic material with an elastic modulus of 10 MPa, estimated from an experimentally derived radial modulus. Contact stresses at interfacial surfaces between the valve and stent were then exported and used to generate applied load estimates.

### 1.2.5.2 FSI Simulation Boundary Conditions

CFD boundary conditions were chosen according to standards established for pediatric heart valve testing for devices deployed on the right side of the heart. The maximum recommended flow parameters corresponding to patients aged between newborn and 4 years were used to simulate realistic worst-case scenarios for the targeted use case of assembling a pediatric BHV. For the systolic configuration, a flow rate of 10 L/min was imposed at both the inlet and outlet. For the diastolic configuration, a 15 mmHg pressure gradient was simulated across the valve, with 25 mmHg used as the pressure proximal to the simulated region for each gradient configuration. Contact between the stent and the PA and the stent and the valve were modeled as bonded contacts, with force measured along the contact region of the stent and the valve. The bonded contact is limited to the inward-facing region of the stent, where microstructures would be incorporated to maintain contact between the stent and the valve. Physiologically, there are additional forces that would be at play between the valve tissue and the PA tissue, which were not incorporated into the model. These forces would reduce the microstructures, so what has been modeled is a worst-case scenario, assuming no other contact forces are at play that contribute to the output loading force.

### 1.2.5.3 Calculation of Fastener Force Distributions For Layout Candidates

Interfacial stresses at all contact nodes between the valve tissue and a single representative strut on the BHV stent were exported from the FSI simulation to estimate the tissue adhesion strength required to safely use MANTIS for assembling the BHV device. Normal and shear stresses at all nodes were linearly interpolated to produce an interfacial stress function σmodel (x,y), with x and z coordinates corresponding to the transverse and longitudinal axes of the strut, respectively. Candidate microstructure layouts consisted of rectangular arrays with m transverse rows and n longitudinal columns, which were evenly distributed over the surface of the strut. The i-th transverse and j-th longitudinal microstructure was assumed to bear the adhesive load of its surrounding rectangular patch A_{i,j}, such that the normal or shear force F_{model}(i,j) mediated by that individual microstructure could be calculated by integrating the corresponding interfacial stress function as follows for the normal loads,

$${F}_{{N}_{model}} ( i , j ) = \int {\int }_{{A}_{i , j}} {σ}_{{N}_{model}} ( x , y ) dA \qquad ( 1 )$$

and for shear loads,

$${F}_{{S}_{model}} ( i , j ) = \int {\int }_{{A}_{i , j}} {σ}_{{S}_{model}} ( x , y ) dA \qquad ( 2 )$$

This calculation was repeated for the tested candidate microstructure layouts containing M={1,2,3} rows and N={40,60,80} columns.

### 1.2.5.4 Safety Factor Calculation And Acceptance Threshold

Safety factors for both normal and shear-loading components were then calculated for each layout candidate. For the normal mode, the mean measured normal pullout strength per microstructure was divided by the maximum simulated normal force applied in any single structure's

$$Normal S . F . = \frac{mean ( {F}_{{N}_{m ax}} )}{max ( {F}_{{N}_{model}} ( i , j ) )} \qquad 3$$

Because shear pullout force measurements were acquired for linear arrays of 42 microstructures, measured maximum shear force values were divided by the number of microstructures in the tested array nₜₑₛₜ=42 to approximate the shear failure force per microstructure. The shear safety factor was obtained by scaling the mean measured maximum shear force per microstructure by the number of microstructure columns n in the simulated layout, then subsequently

$$Shear S . F . = \frac{n \cdot mean ( \frac{{F}_{{S}_{ma x}}}{{n}_{test}} )}{max ( {\sum }_{j = 1}^{n} {F}_{{S}_{model}} ( i , j ) )} \qquad 4$$

Both Normal S.F.>2 and Shear S.F.>2 were required for a given layout to pass the in silico evaluation.
1.2.6 Statistical analysis

All statistical analysis was performed in Python using the Scipy package version 1.11.3. For bending yield testing of single microstructures, the homogeneity of variance was evaluated using Bartlett's test. In the case of nonhomogeneity of variance, Welch's t-test was then used to compare means. Data collected from tissue adhesion tests was assessed for normality by examining the linearity of quantile-quantile relationships between empirical datasets and standard normal distribution. Normal, shear, and peel datasets each deviated from normality, and each dataset was accordingly normalized using a Box-Cox transformation in preparation for parametric statistical analysis. Transformed data was then evaluated for main effects using one-way analysis of variance (ANOVA). A one-sided Dunnett's post-hoc test was used to compare each microstructure design to a relevant control for that experiment. The Flat condition served as the control for normal pullout tests, while the Empty condition served as the control for shear and peel tests. Values in all graphs are expressed as mean±standard deviation. A p-value of <0.05 was considered significant for all statistical tests, with further levels of significance represented in graphs as follows (*: p<0.05; **: p<0.01; ***: p<0.001; ****: p<0.0001).

### 1.3 Results

### 1.3.1 Human Femoral Vein Tissue Characterization

To optimize MANTIS microstructure designs for bioprosthetic heart valve (BHV) assembly, the relevant properties of the human femoral vein (HFV) tissue used in the BHV device were characterized. The anatomical structure and thickness distribution of the HFV wall were examined to inform design decisions regarding microstructure height and geometric profile. Histological analysis of the HFV wall, visualized using Masson's Trichrome stain, revealed the typical multi-layered structure of a blood vessel wall, consisting of the intima at the luminal surface, smooth muscle-rich media, and collagen-rich adventitia. The media layer showed circumferential alignment of ECM and muscle fibers, consistent with expected blood vessel wall anatomy. Full wall thickness was measured using a polymer embedding method, resulting in empirical thickness distributions from 20 sections of HFV samples. The HFV wall thickness followed a log-normal distribution, with values ranging from approximately 180 μm to 3000 μm (FIG. 6). To prevent the MANTIS microstructures from fully puncturing through the HFV wall, the acceptable microstructure height was determined to be less than 180 μm.

### 1.3.2 Design of MANTIS Microstructures

Four distinct microstructured fasteners were designed to compare the performance of multiple geometric strategies. Design inspiration was derived from several macroscale examples, including ancient hunting tools such as arrows and barbed harpoons, as well as bioinspired designs based on the clawed appendage of the praying mantis (FIG. 5A). Ancient weaponry, such as a Bronze Age arrowhead and a Mesolithic harpoon, and the flexing claw of the praying mantis provided insight into efficient piercing and anchoring mechanisms. Microstructures were fabricated from 25.4 μm thick stainless steel sheets using a photochemical machining (PCM) process (FIG. 2), which enabled scalable and rapid manufacturing of the designs for testing. Employing the hook-and-loop principle of operation for probabilistic fasteners, each design featured some re-entrant feature to facilitate entanglement with ECM fibers within the HFV tissue. The Arrow design consisted of a broad tip with barbs on each side, while the Harpoon featured a single barb and a slightly sharper tip than the Arrow. The bioinspired Claw and Jointed Claw designs had wider, hook-shaped microstructures intended to bend upon the application of sufficient compressive force during adhesion. Brightfield micrographs showed these stainless steel MANTIS microstructures, including the Jointed Claw, which contained a notch to provide control over structural deformation behavior, mimicking the jointed foreleg of praying mantis species such as Tenodera sinensis (FIG. 5B). To reduce the probability of microstructures puncturing completely through the HFV wall, the height of all designs was constrained to 150 μm (Table 1.1) in accordance with empirical wall thickness measurements (FIG. 6). Two control designs were also fabricated to enable hypothesis-driven testing of fastener performance: a microstructure-free flat edge and a non-re-entrant microneedle.

TABLE 1.1
nominal dimensions of microstructure designs.

| Feature (μm) | Needle | Arrow | Harpoon | Claw | Jointed Claw |
|---|---|---|---|---|---|
| Base Height | 150 | 150 | 150 | 150 | 150 |
| Base Width | 20 | 20 | 20 | 20 | 20 |
| Feature Height | 50 | 101 | 96 | 103 | 93 |
| Feature Width | 20 | 65 | 60 | 60 | 50 |

1.3.3 Bending yield testing of controllably deformable designs

Before testing adhesion to tissue, bending tests were conducted to characterize the structural yield behavior of the Claw and Jointed Claw designs. It was hypothesized that the notch incorporated within the Jointed Claw design would reduce the bending yield force compared to the Claw. Using a custom mechanical testing instrument (FIG. 7A), individual microstructures were depressed against a rigid glass substrate until structural failure occurred. Force-position curves for each design showed similar deformation stages, starting with a linear elastic response that transitioned into plastic deformation at the yield force (FIG. 7C). Images captured during the tests revealed distinct deformation patterns between the two designs. The Claw bent out-of-plane under compressive load, while the notch in the Jointed Claw facilitated an in-plane bending pattern (FIGS. 7B, 7D). When comparing yield force results (Claw: 87+14 mN; Jointed Claw: 15+1 mN), both yield force (p=0.013) and variance in yield force (p=0.012) were significantly lower for the Jointed Claw compared to the Claw, demonstrating that the notch feature improved control over microstructure deformation as hypothesized (FIG. 7E).

### 1.3.4 Tissue Adhesion Testing

### 1.3.4.1 Normal Pullout of Single Microstructures

To assess the adhesive performance of each MANTIS design, the mechanical interaction between individual microstructures and the adventitial surface of human femoral vein (HFV) tissue was first characterized. A custom mechanical testing instrument designed to measure millinewton scale forces (FIG. 8A) was used to capture both the puncture and pullout stages of the adhesion tests. A slight dip in compressive force was observed during the puncture phase of several tests, although it was unclear whether this signature corresponded to microstructural bending or revealed a discrete puncture event (FIG. 8B). Synchronously captured images showed the re-entrant microstructural features interlocking with HFV tissue fibers, exhibiting robust coupling upon application of tension (FIG. 8C). For example, the controlled deformation behavior of the Claw and Jointed Claw designs enacted a tissue “pinching” effect, in which tissue material became trapped within the confines of the hook-shaped Claw. The adhesive strength was quantified via maximum pullout force F_{Nmax} (FIG. 8B), a metric previously proposed as a standard for comparison between probabilistic fastening systems. F_{Nmax} significantly increased for each re-entrant design (Arrow: 23+21 mN, p<0.01; Harpoon: 29+22 mN, p<0.001; Claw: 49+13 mN, p<0.0001; Jointed Claw: 19+8 mN, p<0.01) compared to the flat control (Flat: 6+7 mN) (FIG. 8D). No significant difference in F_{Nmax} was observed for the Needle design (Needle: 3+1 mN, p=0.95), which lacked re-entrant features (FIG. 8D). While this study design did not incorporate pairwise statistical comparisons between all re-entrant MANTIS designs, F_{Nmax} trended higher for the Claw as compared to the Jointed Claw, suggesting the inclusion of the notch within the Jointed Claw may limit tissue holding strength.

### 1.3.4.2 0° Shear Pullout Of Microstructure Arrays

To further characterize the performance of MANTIS under different loading scenarios and microstructure configurations, adhesive interactions between linear microstructure arrays and the adventitial surface of human femoral vein (HFV) tissues were subsequently measured. Following assembly using an arbor press, tissue adhesion strength of MANTIS microstructure arrays was evaluated in a 0° shear orientation (FIG. 9A). Analysis of synchronous force and image data revealed tight integration between microstructure arrays, with sufficient adhesion to cause cohesive failure of the HFV tissue (FIGS. 9B, 9C). Residual tissue material was frequently observed to remain adhered to microstructure arrays post-test (FIG. 9D). Adhesion strength was quantified via the maximum shear pullout force reached during the test F_{Smax} (FIG. 9B). F_{Smax} significantly increased for each re-entrant design in all orientations (Arrow: 319+157 mN, p<0.0001; Harpoon Primary: 516+179 mN, p<0.0001; Harpoon Secondary: 271+105 mN, p<0.0001; Claw Primary: 892+213 mN, p<0.0001; Claw Secondary: 92+41 mN, p=0.03; Jointed Claw Primary: 569+189 mN, p<0.0001; Jointed Claw Secondary: 101+62 mN, p=0.03) compared to the empty control (Empty: 20+20 mN) (FIG. 9E). Unlike in normal pullout testing, the Needle design exerted significant resistance to shear loading (Needle: 537+251, p<0.0001). The Flat condition lacking microstructures did not significantly differ from the Empty condition (Flat: 121+145 mN, p=0.06). However, outliers were occasionally observed when the tissue became caught on the far corner of the coupon. Interestingly, F_{Smax} for the Claw and Jointed Claw designs trended sharply lower when oriented in the secondary direction compared to the primary direction, suggesting microstructure orientation may affect adhesion performance; however, the current study design precludes statistical conclusions regarding this relationship.

### 1.3.4.3 180° Peel of Microstructure Arrays

To assess adhesion performance in a scenario combining both normal and shear loading components, 180° peel tests were conducted on human femoral vein (HFV) tissues bonded to linear microstructure arrays (FIG. 10A). Contrasting the maximum force approach for quantifying normal and shear adhesion, peel strength was quantified via the metric F_{Pfail}, which captures the force required to cause any significant loss of bond integrity during the test (FIG. 10B). This point of failure appeared as a sudden separation of tissue from the microstructure array at the peel front, accompanied by a peak and subsequent dip in FP (FIGS. 10B, 10C). Excluding the Claw Secondary condition, F_{Pfail} significantly increased for each re-entrant design in all orientations (Arrow: 51±18 mN, p=0.02; Harpoon Primary: 73±64 mN, p<0.01; Harpoon Secondary: 69±59 mN, p<0.01; Claw Primary: 67±43 mN, p<0.01; Claw Secondary: 31±42 mN, p=0.21; Jointed Claw Primary: 101±34 mN, p<0.001; Jointed Claw Secondary: 74+72 mN, p=0.03) compared to the empty control (Empty: 3±1 mN) (FIG. 10D). The Needle design also exerted significant resistance to loading (Needle: 50+12, p=0.02), which may have been caused by some microstructures buckling or bending during the assembly stage. The Flat condition lacking microstructures trended upward in F_{Pfail} but did not significantly differ from the Empty condition (Flat: 15+2 mN, p=0.33). Notably, F_{Pfail} (median=41 mN for re-entrant designs) tended much closer to those observed for F_{Nmax} (median=18 mN for re-entrant designs) than for F_{Smax} (median=310 mN for re-entrant designs), consistent with the concentration of loads on the few microstructures located near the peel front.

### 1.3.5 In Silico Evaluation Of Feasibility For BHV Assembly Application

1.3.5.1 Simulation of in vivo loading conditions.

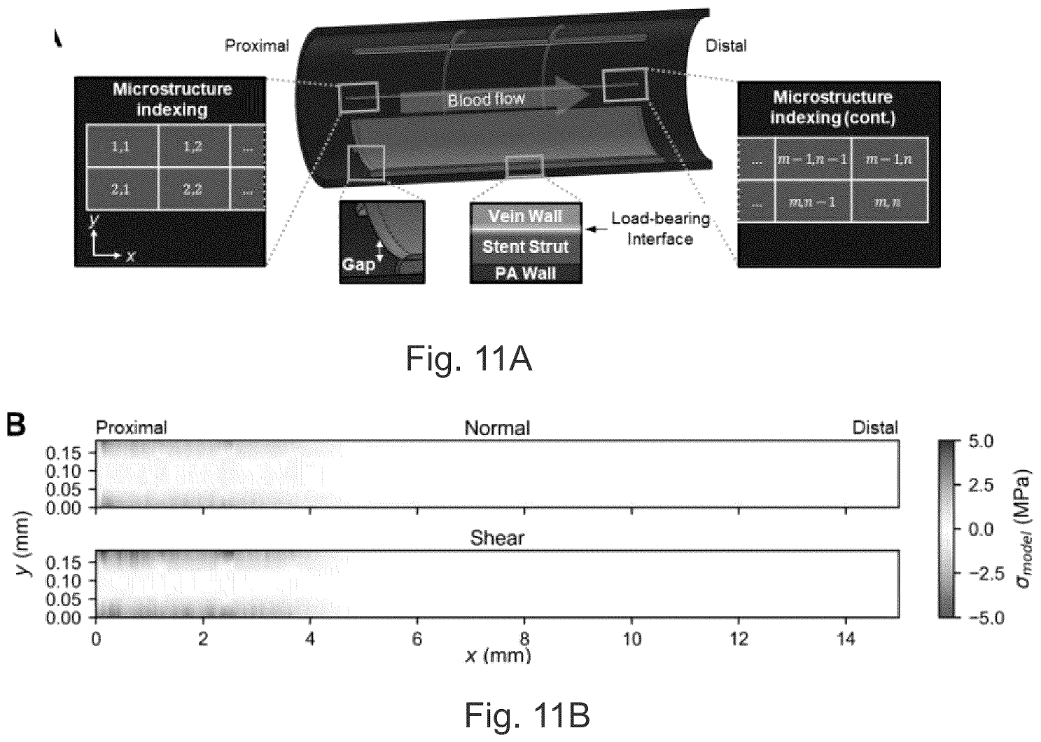

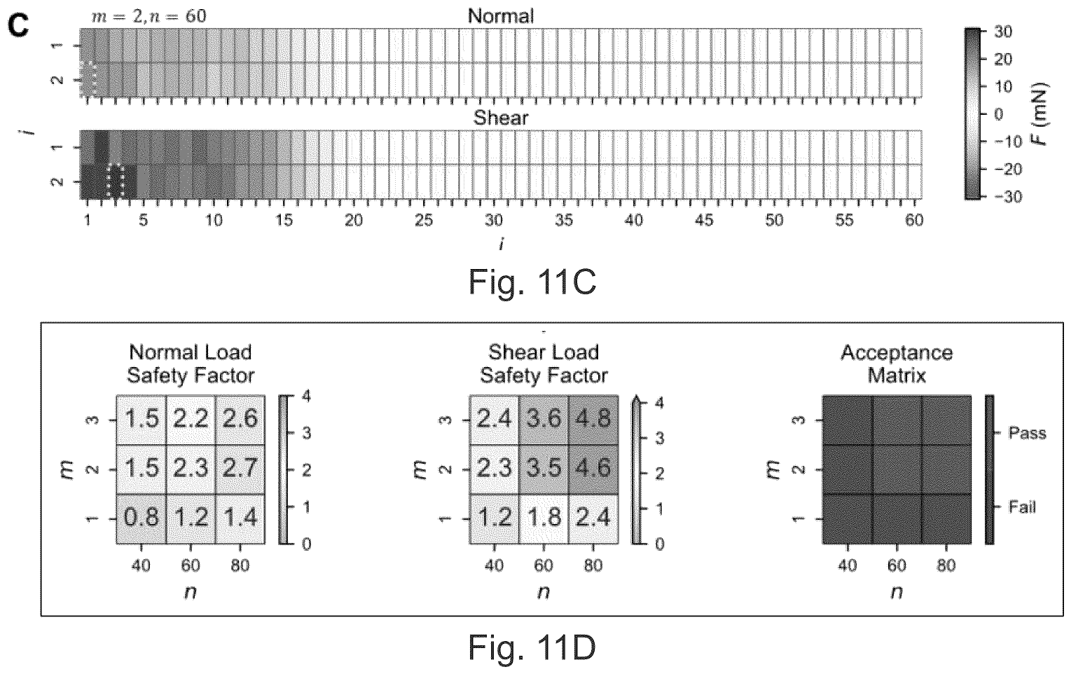

To assess the viability of MANTIS for safely assembling BHVs, FSI simulations of key in vivo BHV operating scenarios were performed to estimate the loads applied at the tissue-stent interface as a result of blood flow through the valve. To evaluate worst-case conditions, model parameters expected to produce the greatest loads were selected, including the maximum flow rates and pressures recommended for testing BHV devices designed for a targeted pediatric patient population. Steady-state solutions for simplified models containing essential geometric features for diastolic and systolic (FIG. 11A) phases of the cardiac cycle yielded stress distributions at the surface of the stent strut, which would hypothetically contain the MANTIS microstructures (FIG. 11B). The largest stresses were observed from the systolic configuration in which fluid flow was permitted to pass between the bioprosthetic valve wall tissue and the PA wall. This paravalvular flow produced a higher concentration of stresses at the edges of the proximal end of the strut than other simulated conditions. All further analysis was performed using this worst-case condition.

1.3.5.2 Topological optimization to identify viable microstructure layouts

To compare the simulated interfacial stresses for the systolic gap configuration against experimental results for MANTIS strength, the tissue-strut interfacial surface was divided into evenly spaced rectangular grids representing theoretical layouts of MANTIS microstructures. A cutaway rendering of the pediatric heart valve stent geometry within the main pulmonary artery (PA) highlights the specific region of the strut where the device would attach, with discretization of axial (i) and circumferential (j) positions along the strut (FIG. 11A). The integration of normal and shear stress functions over the area local to each microstructure produced estimations of the normal and shear loads applied at these positions (FIG. 11C). Interpolated interfacial stress, separated into normal and shear components, revealed substantial variations across different layouts due to the heterogeneous stress distribution (FIG. 11B).

Importantly, the microstructures predicted to bear the maximum loads were identified, and the maximum normal and shear force values for each layout were compared to the experimental mean hold force values for the Claw design, which achieved the highest mean F_{Nmax} and F Smax during tissue adhesion tests (FIG. 11D). Increasing the number of transverse rows from 2 to 3 yielded minimal improvements due to concentrated loads at the strut's edge. A 2×60 layout was found to contain the fewest microstructures while still achieving a safety factor greater than 2 for both normal and shear loading modes. This in silico evaluation, showing calculated force distributions and interpolated stress data, provided critical insights into optimizing microstructure layouts for secure tissue attachment under real-world conditions.

### 1.4 Discussion

The hybrid construction of bioprosthetic heart valves (BHVs) combines the hemocompatibility of a bioprosthetic valve with the structural stability of a metal stent, achieving clinical performance previously unattainable with purely artificial replacement valve designs. However, this composition significantly limits the assembly methods that can be employed, with sutures historically being the only viable option. In this study, a novel alternative to suture-based assembly for BHV devices is proposed using MANTIS, a microstructured probabilistic fastening system designed for the rapid attachment of metal components to biological tissues. A family of four distinct fastener types was generated by drawing on a rich 90,000-year history of human innovation in barbed tool design and biomimicry of the praying mantis claw's prey capture function. Coupling this design approach with a well-established PCM fabrication process yielded microstructures with excellent tissue adhesion performance, with all four designs achieving significant improvement over controls in normal, shear, and peel loading modes.

Probabilistic fastener design must begin with careful consideration of the material and structural properties of the mating surface to ensure successful adhesion. In this case, the mating surface is the adventitial side of a naturally occurring venous valve graft, a multi-layered tissue whose mechanical integrity primarily derives from collagen and elastin protein fibers in the adventitia and media layers, respectively. The aim was to design microstructured fasteners capable of readily puncturing and interlocking with this fibrous matrix to form secure connections that resist multi-directional loads. The PCM fabrication process enabled achievement of both these critical functions, producing high aspect ratio, complex microscale geometries (Table 1.1) with sharp tips, re-entrant barbs, and deformable joints (FIG. 5B). It appears that the Jointed Claw fastener presented here is the smallest implementation of controlled deformation in a tissue fastener reported to date, reaching a closed height below 100 μm while the smallest commercially available surgical staples reach a closed height of 750 μm. Additionally, the arrayed microstructure layouts presented here were primarily configured for the characterization of adhesion performance, and a PCM approach may produce arbitrary spacings and orderings of different fastener types to yield more optimal macroscale adhesion characteristics.

The objective was to design microstructured fasteners that could easily puncture and interlock with the fibrous matrix, creating secure connections capable of resisting multi-directional loads. Normal pullout tests revealed the processes of puncture and pullout for individual microstructured fasteners, the fundamental units of the MANTIS system (FIG. 8B). When assuming linearly scaled load sharing by multiple fastener elements, the effective adhesive strength of many fasteners is expected to tend toward the average behavior of a single fastener. As a result, testing individual fasteners should also provide the best measure of variance in adhesion strength. The results for F_{Nmax} span ranges of up to ˜12-fold for a given design (e.g., 5 mN-61 mN for Arrow), indicating considerable variability in the quality of tissue integration. However, the reductive nature of individual fastener tests neglects any nonlinear scaling effects that may exist for the MANTIS microstructure-HFV tissue system. Conversely, shear tests performed with microstructure arrays more closely mimic the BHV assembly use case at the expense of concealing variation in individual structure contributions along the array. Furthermore, across all tissue adhesion test types, a nontrivial challenge exists in distinguishing adhesive failure processes of the MANTIS bond from cohesive failure processes of the tissue material. F_{Nmax} and F_{Smax} were usually recorded well after permanent destruction of the tissue material had already occurred (FIGS. 8C, 9C). Blood vessel wall biomechanical behavior is complex, incorporating hyperelastic stress-strain relations, viscoelastic relaxation responses, and failure behavior dependent on multi-scale fiber pullout processes.

To contextualize the mechanical performance of MANTIS against established suture-based approaches, analogous tests of the single structure normal pullout test were performed on sutures (supplemental materials). The loop connecting the suture to the testing apparatus was the consistent failure point with observed tensile failure forces (2-3 N) comparable to the manufacturer's reported straight tensile strength (4.7 N). Suture retention results are highly prone to testing configurations such as suture diameter, knot type, and depth of bite.

Finally, as a proof of concept for integrating MANTIS into the BHV device assembly process, the approach's feasibility was evaluated in silico. In simulations of in vivo fluid-structure interaction (FSI) mechanics of the BHV, realistic model parameters were consistently chosen to produce worst-case loading scenarios. The simulations identified paravalvular flow between the stent-valve graft wall and the pulmonary artery (PA) lumen as a critical factor, driving a ˜50× increase in interfacial stress magnitudes compared to other geometries. Despite significant stress concentration at the proximal edges of the stent strut surface caused by this flow, the top-performing Claw design successfully reached a viable safety factor in several microstructure layouts obtained via a simple topological optimization process. Topological optimization methods have previously been implemented to tune microscale distributions of adhesive strength to realize improved macroscale characteristics. While the optimization was constrained to regularly spaced rectangular grids of a single fastener design, more sophisticated optimization approaches may relax these constraints to yield a higher safety factor, in addition to accounting for the probabilistic nature of fastener attachment strength. More broadly, this design, test, and simulation methodology may be used to evaluate the potential of MANTIS to perform assembly or implantation functions for applications beyond BHVs such as wound closure. Limitations of the presented modeling approach include the use of simplified geometric representations of the BHV device in both systolic and diastolic configurations, which may not capture forces propagated by valve leaflets. Additionally, while steady state simulations are expected to correspond to instances of maximal loading, they fail to track the dynamic transitions between loading states which would occur throughout the cardiac cycle.

### 1.5 Conclusions

In these experiments, several metallic microstructured fasteners with re-entrant profiles were designed, demonstrating strong adhesion to thin compliant tissue under normal, shear, and peel loads. The implementation of controlled deformation in the Jointed Claw design is a particularly novel development, representing the most compact application of such functionality for tissue adhesion reported to date. FSI modeling of BHV operation suggests rectangular arrays of MANTIS microstructures could withstand forces that will be experienced in vivo. In summary, MANTIS demonstrates excellent potential for translation as a sutureless tissue adhesion method that addresses challenges in BHV assembly and beyond.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A micro-structured mechanical adhesive comprising:
a base section having a first side and an opposite second side; and
a plurality of microstructures extending from the first side of the base section, each microstructure configured to yield in a predetermined direction upon adhesion to biological tissue and to provide attachment to biological tissue.

2. The micro-structured mechanical adhesive of claim 1, wherein the plurality of microstructures include a microstructure selected from the group consisting of hooks, pillars, and posts.

3. The micro-structured mechanical adhesive of claim 1, wherein the plurality of microstructures includes hooks that are curved as they extend from the first side of the base section.

4. The micro-structured mechanical adhesive of claim 3, wherein each hook tapers as it extends from the first side of the base section.

5. The micro-structured mechanical adhesive of claim 1, wherein each microstructure includes barbs extending therefrom.

6. The micro-structured mechanical adhesive of claim 1, wherein each microstructure includes a hook and wherein said hook defines a preferential failure notch that biases the hook to yield in the predetermined direction.

7. The micro-structured mechanical adhesive of claim 6, wherein the preferential failure notch defines a point of structural failure to bias the hook to yield in the predetermined direction by a threshold force.

8. The micro-structured mechanical adhesive of claim 6, wherein the base section and the plurality of microstructures are unitary.

9. The micro-structured mechanical adhesive of claim 1, wherein each microstructure is characterized by a yield force that is adjustable by varying a notch depth.

10. The micro-structured mechanical adhesive of claim 1, wherein each microstructure is characterized by a yield force that is adjustable by varying length proportions of sections below and above a notch.

11. The micro-structured mechanical adhesive of claim 1, wherein the base section and the plurality are composed of nitinol or stainless steel.

12. The micro-structured mechanical adhesive of claim 1, wherein the base section and the plurality are composed of a polymer.

13. The micro-structured mechanical adhesive of claim 1, wherein each microstructure has a length from about 50 to 250 microns.

14. The micro-structured mechanical adhesive of claim 1, wherein each microstructure has a base proximal to the base section, and where a width at the base is from 10 to 50 microns.

15. The micro-structured mechanical adhesive of claim 1, wherein a preferential failure notch reduces a bending yield force.

16. The micro-structured mechanical adhesive of claim 15, wherein the bending yield force is less than 25 mN.

17. A method of attachment with a micro-structured mechanical adhesive comprising a base section having a first side and a second side and a plurality of microstructures extending from the first side of the base section, each microstructure configured to yield in a predetermined direction upon adhesion to biological tissue and to provide attachment to biological tissue, the method comprising:
attaching the micro-structured mechanical adhesive to a biological tissue such that the plurality of microstructures extend into the biological tissue to form a mechanical connection.

18. The method of claim 17, wherein the micro-structured mechanical adhesive is attached to a medical device.

19. The method of claim 17, wherein bending of the microstructures provides mechanical integration with the biological tissue.

20. A growth-adaptive stent for use in a heart valve system comprising:
a stent body defined by a plurality of struts and a plurality of spring elements, the plurality of struts providing structural support, wherein the spring elements are arranged between the struts and configured to flex and expand between adjacent struts, wherein the struts include slot features for attaching a micro-structured mechanical adhesive.

21. A growth-adaptive heart valve prosthetic device comprising:
a valve body configured to be implanted in a heart, the valve body having valve leaflets for regulating blood flow, the valve body composed of naturally occurring venous valve tissue; and
a growth-adaptive stent attached to the valve body, the growth-adaptive stent comprising a plurality of spring elements attached to a plurality of struts that provide structural support, wherein the spring elements are configured to flex and expand, allowing the growth-adaptive stent to adapt to growth of a patient's heart.

22. The growth-adaptive heart valve prosthetic device of claim 21, wherein the growth-adaptive stent is attached to the valve body with a micro-structured mechanical adhesive configured to attach the growth-adaptive stent to cardiac tissue.

23. The growth-adaptive heart valve prosthetic device of claim 22, wherein the micro-structured mechanical adhesive comprises re-entrant microstructure geometries forming a mechanical interlock between the valve body and the growth-adaptive stent, designed to minimize local stress concentrations and reduce tissue tearing.

24. The growth-adaptive heart valve prosthetic device of claim 22, wherein the micro-structured mechanical adhesive includes a base section having a first side and a second side, and a plurality of microstructures extending from the first side of the base section, each microstructure configured to preferentially yield or buckle in a predetermined direction, wherein the valve body and the growth-adaptive stent are configured to provide continuous valve functionality over a predetermined expansion range, maintaining proper leaflet motion and blood flow regulation at all diameters.

25. The growth-adaptive heart valve prosthetic device of claim 21, wherein the growth-adaptive stent is composed of a superelastic material providing a low chronic outward force that allows for controlled expansion in response to growth of surrounding tissue.
